(19) United States
(12) Patent Application Publication
(10) Pub. No.: US 2016/0015379 A1
(43) Pub. Date: Jan. 21, 2016

(54) Curvilinear Transosseous Rotator Cuff Repair Tools

(71) Applicant: MicroAire Surgical Instruments, LLC, Charlottesville, VA (US)

(72) Inventors: Douglas Keller, Palmyra, VA (US); Ryan Vanleeuwen, Ruckersville, VA (US); Brian D. Dross, Mount Pleasant, SC (US)

(21) Appl. No.: 14/560,668

(22) Filed: Dec. 4, 2014

Related U.S. Application Data
(63) Continuation-in-part of application No. 14/331,304, filed on Jul. 15, 2014.

(51) Int. Cl. A61B 17/04

(52) U.S. Cl. CPC A61B 17/0469; A61B 2017/0472; A61B 2017/0477

(57) ABSTRACT

Rotator cuff tears (complete and partial) are surgically repaired without needing more dermal access than percutaneous punctures. The surgeon is afforded a range of options for where to place a second tunnel in the bone, and selection by the surgeon of a desired bone bridge size is provided in advance of tunnel construction. Also, stitching in rotator cuff surgery can be accomplished without the need for suture anchors or added cost of secondary suture passers. Multiple stitch configurations including X-patterns are possible with combinations of preloaded loops and or sutures passed simultaneously through tissue and bone. Fixation is achieved without the use of suture anchors, buttons or other rigid implants.

### FIELD OF THE INVENTION

The invention relates to surgical methods and surgical tools, especially for rotator cuff surgery.

### BACKGROUND OF THE INVENTION

In connection with surgery for rotator cuff repair, certain surgical tools and technology are known. The patent literature includes certain work regarding rotatory cuff surgery, mentioned in roughly chronological order as follows:

U.S. Pat. No. 6,514,274 issued Feb. 4, 2003 to Boucher et al. (Arthrotek, Inc.), for “Method and apparatus for rotator cuff repair.”

U.S. Pat. No. 7,569,059 issued Aug. 4, 2009 to Cerundolo (Arthroscopic Innovations LLC) for “Method and apparatus for surgical repair.”

U.S. Pat. No. 7,604,659 issued Oct. 20, 2009 to Lee, for “Method and apparatus for repair of torn rotator cuff tendons.”

U.S. Pat. No. 7,771,441 issued Aug. 10, 2010 to Cerundolo for “Method and apparatus for providing suture in a passageway.”

U.S. Pat. No. 7,833,230 issued Nov. 16, 2010 to Cerundolo for “Method and apparatus for providing a passageway.”

U.S. Pat. No. 7,955,341 issued Jun. 7, 2011 to Cerundolo for “Method and apparatus for providing suture in a passageway.”

U.S. Pat. No. 8,016,883 issued Sep. 13, 2011 to Coleman et al. (Tepha, Inc.), for “Methods and devices for rotator cuff repair.”

U.S. Pat. No. 8,114,127 issued Feb. 14, 2012 to West, Jr. (HS West Investments, LLC), for “Bone anchors for use in attaching soft tissue to bone.”

U.S. Pat. No. 8,277,458 issued Oct. 2, 2012 to Schneider (Biomet Sports Medicine, LLC), for “Apparatus and method for arthroscopic transhumeral rotator cuff repair.”

U.S. Pat. No. 8,282,643 issued Oct. 9, 2012, to Dross (ION Surgical Technologies, Inc.), for “Arthroscopic Method and Apparatus for Tissue Attachment to Bone.”

U.S. Pat. No. 8,409,225 issued Apr. 2, 2013 to Bull et al. (Medical Device Innovations Ltd.), for “Tendon repair.”

U.S. Pat. No. 8,435,294 issued May 7, 2013 to Montgomery et al. (Cayenne Medical, Inc.), for “Devices, systems and methods for material fixation.”

U.S. Pat. No. 8,460,318 issued Jun. 11, 2013 to Murray et al. (Arthrex, Inc.), for “Suturing instrument and method for passing multiple sutures.”

U.S. Pat. No. 8,523,903 issued Sep. 3, 2013 to Kilburn-Peterson et al. (DePuy Mitek LLC), for “Partial thickness rotator cuff repair system and method.”

U.S. Pat. No. 8,529,601 issued Sep. 10, 2013 to Green et al. (KF x Medical Corp.), for “System and Method for Attaching Soft Tissue to Bone.”

U.S. Pat. No. 8,540,737 issued Sep. 24, 2013 to Chudik, for “Method of arthroscopic or open rotator cuff repair using an insertional guide for delivering a suture pin.”

U.S. Pat. No. 8,545,535 issued Oct. 1, 2013 to Hirotsuka et al. (Foundry Newco XI, Inc.), for “Suture anchors with one-way cinching mechanisms.”

U.S. Pat. No. 8,556,911 issued Oct. 15, 2013 to Mehta et al., for “Arthroscopic tunnel guide for rotator cuff repair.”

U.S. Pat. No. 8,597,336 issued Dec. 3, 2013 to van der Burg, et al. (Howmedica Osteonics Corp.), for “Apparatus for discrete tissue anchoring for soft tissue repair and method of use.”

U.S. Pat. No. 8,617,186 issued Dec. 31, 2013 to White et al. (ArthroCare Corp.), for “Independent suture tensioning and snaring apparatus.”

U.S. Pat. No. 8,672,967 issued Mar. 18, 2014 to DiMatteo et al. (DePuy Mitek LLC), for “Partial thickness rotator cuff repair system and method.”

U.S. Pat. No. 8,696,703 issued Apr. 15, 2014 to Anspach, III, et al. (DePuy Synthes Products, LLC), for “Anchor/suture used for medical procedures.”

Even with all of the work relating to rotator cuff surgery, still there are shortcomings of, and room for improvement over, the existing technology.

For example, one problem with much existing technology used in conventional rotator cuff surgery is that typically at least one relatively-big dermal incision must be made in the shoulder.

Another shortcoming of existing surgical tools is that a surgeon's need to know where a tunnel is created when the tool is operated was not being met, for example, the surgeon had to tolerate the medial tunnel appearing wherever the tool created the tunnel and the surgeon was unable to plan where the medial tunnel would be created.

Another problem with existing tools used for rotator cuff surgery is that the surgeon is not given a range of options for where the second tunnel can be formed in the bone and correspondingly, when the conventional tools are used and the second tunnel is formed, a bone bridge that results is not necessarily of desirable dimensions or geometry, and often is a thinner bone bridge than would be desirable.

### SUMMARY OF THE INVENTION

Certain surgical tools, devices and methods have been invented which are particularly useful for rotator cuff surgery, especially for facilitating rotator cuff surgery without needing surgical incisions to be as large as with previous surgical technology and/or providing the surgeon with a range of options for where tunnels are formed in bone, so that the surgeon has more control over the bone bridge that is being formed when the two tunnels are constructed.

In one preferred embodiment, the invention provides a surgical tunneling tool, comprising: a first member (such as, e.g., a first member that is relatively straight and vertical); a second member (such as, e.g., a second member that is curved); a support member that defines a separation between the first member and the second member (such as, e.g., a separation between the first member and the second member in a range of about 25-85 degrees; a separation wherein the first member is at a 12 o'clock position and the second member is at about a 3 o'clock position; etc.); a medial tunneler that extends inward from the first member, wherein the medial tunneling member is straight; and, a lateral tunneler that extends inward from the second member, wherein the lateral tunneler is curved; such as, e.g., an inventive tool further comprising a syringe-shaped cavity; an inventive tool further comprising a fluid connection in a vicinity of a syringe-shaped cavity; an inventive tool comprising an obturator fitted to the first member; an inventive tool comprising a K-wire fitted to the first member; an inventive tool comprising a drill; and other inventive tools.

The invention in another preferred embodiment provides a surgical tunneling tool that when operated forms a medial tunnel and a lateral tunnel, comprising: a medial tunneler; a lateral tunneler; and a bone bridge indicator having multiple positions and that shows in advance what bone bridge between the medial tunnel and the lateral tunnel will be created, wherein a bone bridge indication displayed on the indicator is a function of a current position of the medial tunneler (such as, e.g., a bone bridge planning indicator that comprises a set of markings ranging from a minimal bone bridge up to a 3 cm bone bridge); such as, e.g., an inventive tool wherein indication markers are on a medial cannula; an inventive tool further comprising bone bridge indicator markings on a medial cannula, wherein the bone bridge indicator markings correspond to an actual constructed bone bridge with an accuracy of about 10 mm or better (such as, e.g., bone bridge indicator markings that correspond to the actual constructed bone bridge with an accuracy of about 5 mm or better); and other inventive tools.

In another preferred embodiment, the invention provides a method of tunneling in shoulder surgery, comprising: constructing a medial tunnel using a versatile surgical tool that comprises a guide body, a medial tunneler and a top handle, wherein the medial-tunnel-constructing is performed according to a medial-tunnel-forming process selected from the group consisting of: a) a K wire process comprising forming the medial tunnel by use of a K wire, followed by sliding the guide body of the surgical tool over the K wire; b) a drill guide process comprising, with the guide body positioned as a drill guide, inserting a drill bit through the guide body and drilling the medial tunnel; and c) a mallet action process comprising applying force to the top handle of the surgical tool to mallet the medial tunneler in and thereby form the medial tunnel.

The invention in another preferred embodiment provides a surgical method during repair of a rotator cuff in a vicinity of a humeral head, comprising: while positioning the rotator cuff, holding the rotator cuff to the humeral head (such as, e.g., a holding step performed by a pointed tip of an obturator of the surgical tool), wherein a same surgical tool performs the positioning and the holding; such as, e.g., inventive surgical methods further comprising piercing a to-be-repaired tendon with a pointed tip of an obturator, followed by pulling the pierced tendon to a desired location, followed by constructing a tunnel; inventive surgical methods further comprising constructing a tunnel with a to-be-repaired tendon in place, and without usage of a soft tissue suture passer device.

In another preferred embodiment the invention provides a surgical tunneling method, comprising: medial tunneling in a shoulder area of a patient, wherein a straight medial tunnel is formed by a medial tunneler, followed by lateral tunneling in the shoulder area, wherein a curved lateral tunnel is formed by a lateral tunneler, wherein the medial tunneler and the lateral tunneler are provided on a same surgical tool; such as, e.g., inventive tunneling methods wherein a formed location of the medial tunnel after the tool has been operated by the surgeon to form the medial tunnel matches a preplanned location that the surgeon intended the medial tunnel to define; inventive tunneling methods wherein the medial tunneling by the medial tunneler is at a place where a pin has been placed, before the medial tunneling step, wherein the pin represents where the surgeon operating the surgical tool wants the medial tunnel to be formed; inventive tunneling methods wherein the medial tunnel is formed by the surgical tool in a place preselected by the surgeon before tunnel-formation, followed by the lateral tunnel being formed by the surgical tool in a place preselected by the surgeon before tunnel-formation; and other inventive methods.

The invention in another preferred embodiment provides a surgical tunneling method performed by operation of a surgical tool that comprises a medial tunneler and a lateral tunneler, the method comprising: forming a medial tunnel by operation of the medial tunneler; after the surgical tool has been adjusted, forming a lateral tunnel by operation of the lateral tunneler; such as, e.g., inventive surgical tunneling methods further comprising (1) adjusting depth of the medial tunnel and (2) rotating the surgical tool, thereby selecting a location of where the lateral tunnel will be formed.

In another preferred embodiment the invention provides a method of formation of a lateral tunnel and a medial tunnel during rotator cuff surgery, comprising: forming the lateral tunnel via access that is a percutaneous needle stick; forming the medial tunnel via access that is a percutaneous needle stick.

The invention in another preferred embodiment provides a suture passer kit, comprising: a suture retriever comprising a first member (e.g., a first member that is a tube (e.g., a plastic tube; etc.)) with a loop (e.g., a wire loop) at a distal end of the first member; and a second member (e.g., a second member that is a tube (e.g., a plastic tube, etc.)) having assembled thereon a set of sutures (e.g., a set of sutures that includes at least two different-colored sutures)), wherein the second member is sized to fit inside a medial tunnel, and wherein the second member and/or the sutures thereon are pullable by the loop on the suture retriever; such as, e.g., an inventive suture passer kit wherein the first member comprises an elongated member and the second member comprises an elongated member; an inventive suture passer kit wherein the second member is pullable by the loop on the suture retriever when the loop captures the suture passer at an intersection where the medial tunnel intersects a lateral tunnel; an inventive kit wherein the first member is a plastic tube and the second member is a plastic tube; an inventive kit in which suture anchors are not contained nor used with the kit; etc.

In another preferred embodiment, the invention provides a surgical stitching method during rotator cuff repair, comprising: surgically constructing a set of tunnels (such as, e.g., one tunnel; two tunnels; three tunnels; etc.), followed by stitching without use of suture anchors and comprising use of a preloaded suture passer kit; such as, e.g., inventive surgical stitching methods wherein when the stitching step has been completed, a stitch pattern is formed selected from the group consisting of: a) a simple stitch pattern stitching a single tunnel; b) a box stitch pattern with inverted mattress stitch with knotless medial row stitching a set of two tunnels; c) a box stitch pattern with inverted mattress stitch with a locking medial row stitching a set of two tunnels; d) a standard X stitch pattern stitching a set of two tunnels; e) a boxed-X stitch pattern stitching a set of two tunnels; f) a stitch pattern stitching a set of three tunnels, and wherein no suture anchor is present; inventive methods comprising, for a set of stitches stitched during the stitching step, securing the stitches with knots in or under a cortical layer of bone; inventive methods comprising, for a set of stitches stitched during the stitching step, securing the stitches with knots not in a cortical layer of bone; inventive methods comprising, for a set of stitches stitched during the stitching step, securing the stitches with Tri-Calcium Phosphate (TCP) or other bone cement, with or without knots (such as, e.g., securing the stitches with TCP performed supplemental to knot-tying; securing the stitches with TCP performed without knot tying); and other inventive methods.

Another preferred embodiment of the invention provides a surgical stitch pattern, comprising: a set of sutures defining a boxed-X shape, wherein the sutures stitch a surgical site (such as, e.g., a surgical site that is a shoulder) on a patient; such as, e.g., an inventive surgical stitch pattern wherein the boxed-X shape of the set of sutures compresses tissue against bone; an inventive surgical stitch pattern wherein a partially-torn rotator cuff is stitched; an inventive surgical stitch pattern wherein a fully-torn rotator cuff is stitched; and other inventive surgical stitch patterns.

The invention in another preferred embodiment provides a surgical method, comprising: forming a tunnel, wherein the tunnel-forming is performed by a surgical tool (preferably a surgical tool of this invention); aspirating biologic material from where the tunnel-forming is performed, wherein the surgical tool that performs the tunnel-forming is used to perform the aspirating without using a separate surgical tool for aspirating; such as, e.g., inventive surgical methods wherein the tunnel-forming and aspirating are in a shoulder; and other inventive surgical methods.

In another preferred embodiment, the invention provides a method of holding a withdrawal device (such as, e.g., a syringe) while the withdrawal device is receiving biologic material being withdrawn from a shoulder area of a patient during a surgical procedure, comprising: receiving the withdrawal device into a section of a tunnel-forming device (preferably a tunnel-forming device of this invention); and holding the withdrawal device while biologic material is being received therein, wherein the holding is performed by the tunnel-forming device; such as, e.g., inventive methods wherein a syringe is connected by a fluid connection on the tunnel-forming device; inventive methods wherein the holding step is performed by the tunnel-forming device while the withdrawal device withdraws biologic matter from a humeral head of the patient; inventive methods wherein the holding step is performed by the tunnel-forming device while the withdrawal device aspirates bone marrow from a bone into which a tunnel is being formed by the tunnel-forming device; inventive methods comprising forming the tunnel through bone; and other inventive methods.

Also in a preferred embodiment the invention provides a surgical tunnel-forming device comprising: a first member having a bottom section from which protrudes a straight tunneler; and a fluid connection positioned on the first member and compatible with a syringe; such as, e.g., an inventive device further comprising the syringe held onto the device by the fluid connection; an inventive device further comprising a lateral member from which protrude a curved tunneler, wherein the straight tunneler and the curved tunneler are in intersecting relationship; an inventive device wherein the straight tunneler and the curved tunneler are respectively operable when a syringe is connected; and other inventive devices.

### BRIEF DESCRIPTION OF THE DRAWINGS

The invention may be appreciated by reference to the figures, which are not necessarily drawn to scale:

FIG. 1 is a perspective view of an exemplary inventive surgical tunneling tool 1.

FIG. 1A is a diagrammatic perspective view of an exemplary inventive surgical tunneling tool 1′.

FIG. 1B depicts arcuate lumen 7 of tool 1.

FIG. 1C depicts lateral tunneler 6 of tool 1.

FIG. 1D depicts luer lock collar 19 of tool 1.

FIG. 1E depicts guide body 11 of tool 1.

FIG. 1F depicts medial tunneler 5 of tool 1.

FIG. 2 is a perspective view of an inventive surgical tunneling tool in which the handle comprises harvest port 9 into which is received syringe 10 which is attachable and detachable from the tool.

FIG. 2A is a cross-sectional view of part of medial tunneler 5 and FIG. 2B is an enlarged view of the a section of FIG. 2A depicting luer lock collar 19.

FIG. 3 depicts tool 1 (FIG. 1) in an exploded perspective view.

FIG. 3A is a cross-sectional view of tool 1 (FIG. 1).

FIG. 4 is a perspective view of a suture passer 16 according to an embodiment of the invention, threaded with suture 17.

FIGS. 4A-4B are projected views of suture passer 16.

FIG. 4C is a perspective view of suture passer 16, according to another embodiment of the invention.

FIG. 5 is a cross-sectional view relating to a grasp-and-pierce method step for tissue approximation according to an exemplary embodiment of the invention.

FIG. 5A is a cross-sectional view relating to a pierce-and-relocate method step for tissue approximation according to an exemplary embodiment of the invention.

FIG. 6 is a cross-sectional view relating to a medial tunnel formation step according to an exemplary embodiment of the invention.

FIG. 7 is a cross-sectional view relating to a lateral tunnel initiation step according to an exemplary embodiment of the invention.

FIG. 8 is a cross-sectional view relating to a lateral tunnel completion step according to an exemplary embodiment of the invention.

FIG. 9 is a cross-sectional view relating to a suture passing preparation step according to an exemplary embodiment of the invention.

FIG. 10 is a cross-sectional view relating to a suture loading/passing step according to an exemplary embodiment of the invention.

FIG. 11 is a cross-sectional view relating to a component removal step according to an exemplary embodiment of the invention.

FIGS. 12-12A are diagrams of repaired tendon after repair using an exemplary embodiment of the invention. FIG. 12 shows a locking medial row and lateral subcortical knot construct that minimizes micro-motion and lateral bone stress. FIG. 12A shows a knotless medial row and a subcortical knot construct that minimizes muscle tendon junction stress, subacromial impingement and lateral bone stress.

FIGS. 13-18E are diagrams of exemplary stitch patterns according to various embodiments of the invention, including suture management for a single tunnel, with a simple stitch (FIGS. 13-13C); suture management for two tunnels, with box/inverted mattress with a knotless medial row (FIGS. 14-14D); suture management for two tunnels, for box/inverted mattress with a locking medial row (FIGS. 15-15E); suture management for two tunnels, with a standard “X” stitch pattern (FIGS. 16-16E); suture management for two tunnels, with a boxed-X shaped stitch pattern (FIGS. 17-17E); and suture management for three tunnels (FIGS. 18-18E).

FIG. 19 is a perspective view showing bone bridge markings on a tool according to an exemplary embodiment of the invention.

FIGS. 20-20A are projected views of suture retriever 15 useable in practicing an exemplary embodiment of the invention.

FIG. 21 is a projected view of another exemplary embodiment of an inventive tool, that is laterally hinged.

FIG. 22 is a perspective view of a suture passer 216 (see Example 5A) according to another exemplary embodiment.

FIG. 22A is a side view of suture passer 216 of FIG. 22.

FIG. 22B is a closeup X-ray view of FIG. 22A showing coiled wire 203.

FIG. 22C is a cross-section of FIG. 22B showing coiled wire 203.

FIG. 23 is an exploded perspective view of a removable medial cannula 2305 having a distal luer connection according to another exemplary embodiment of the invention. Each of FIGS. 23A, 23B, 23C is a perspective view of an alternative example of tip 2300 in FIG. 23.

FIG. 24 (see Example 7) is a perspective view of a luer tip/cannula component according to another exemplary embodiment of the invention.

FIG. 24A is a rotated view of part of suture passer 2416 of FIG. 24.

FIG. 24B is a cross-section of FIG. 24A.

### DETAILED DESCRIPTION OF A PREFERRED EMBODIMENT

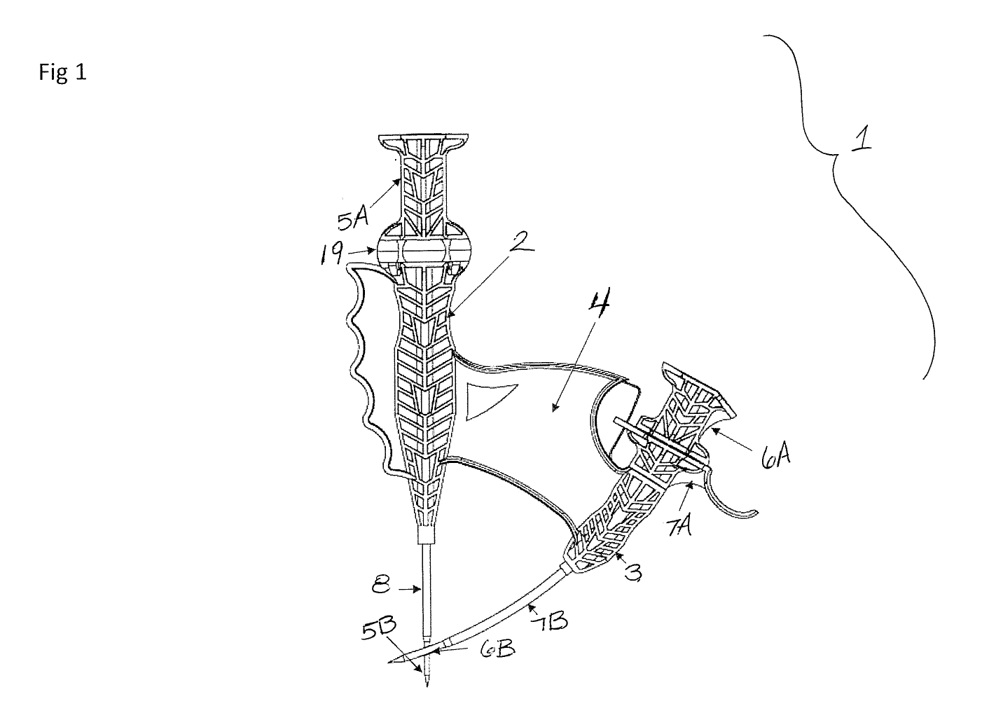

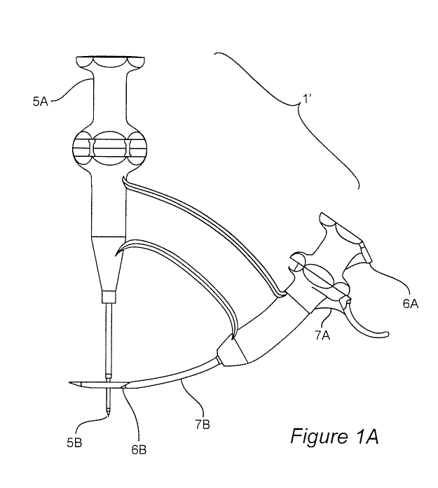

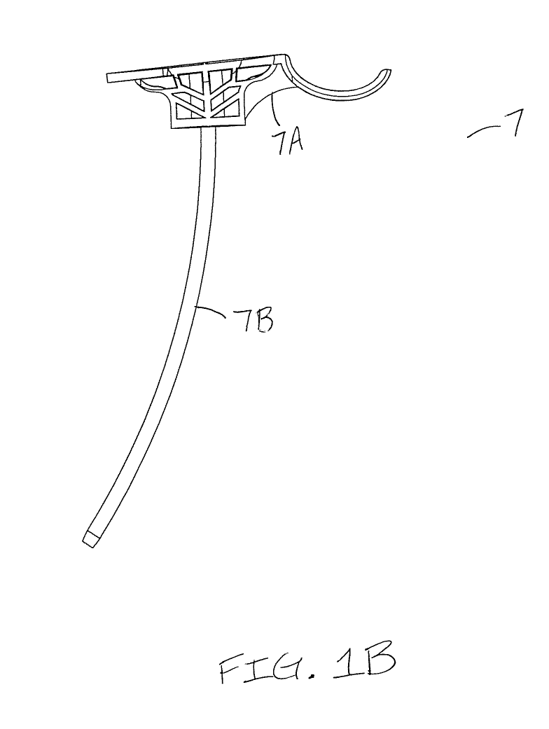

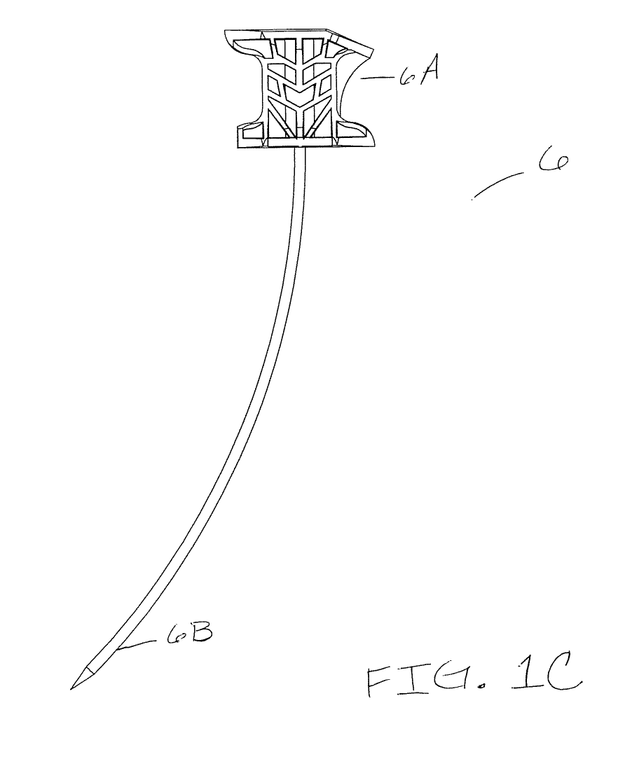

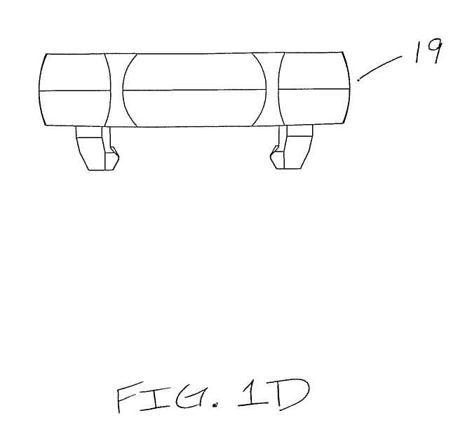

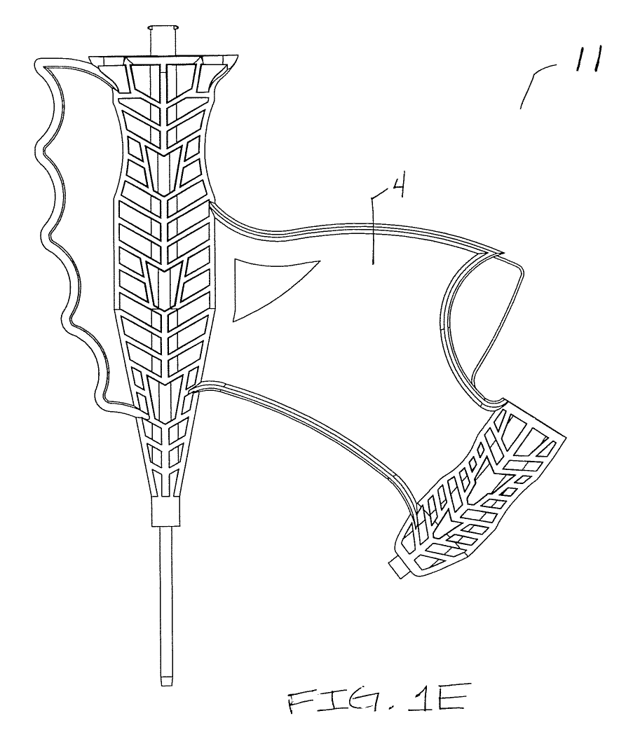

The invention addresses certain shortcomings and limitations of existing technology used in rotator cuff repair surgery, in which bone-tunneling must be performed. Surgical tunneling tools (such as, e.g., surgical tunneling tool 1 of FIG. 1; surgical tunneling tool 1′ of FIG. 1A) according to the invention improve upon existing surgical tool technology.

Surgical tunneling tool 1 comprises a first member 2 (preferably a member that is relatively straight and vertical) and a second member 3 (preferably a curved member). A support member 4 defines a separation between the first member 2 and the second member 3. For a separation between the first member 2 and the second member 3, preferably the separation is in a range of about 25-85 degrees.

A medial tunneler 5 extends inward from the first member 2. Medial tunneler 5 comprises handle 5A and obturator tip 5B. Medial tunneling member 5 is straight. Medial tunneling member 5 is used to create a straight tunnel. Advantageously tool 1 is useable to create a medial tunnel (straight tunnel) first, BEFORE another tunnel (i.e., the curved tunnel) is created.

A lateral tunneler 6 extends inward from the second member 3. The lateral tunneler 6 is curved.

Tunnelers 5, 6 are in intersecting relationship, and correspondingly, a pair of intersecting bone tunnels is formable by operation of tunnelers 5, 6.

Preferably tool 1 comprises a fluid connection in a vicinity of first member 2.

Lateral tunneler 6 is disposed inside an arcuate lumen 7. Lateral tunneler 6 comprises handle 6A and obturator tip 6B. Arcuate lumen 7 comprises handle 7A and arcuate tube 7B.

Medial tunneler 5 is disposed inside a straight lumen 8. Preferably straight lumen 8 is integrated in first member 2.

Tool 1 is useable to form a medial tunnel and a lateral tunnel in bone.

Preferably tool 1 comprises a bone bridge planning indicator (not shown in FIG. 1) having multiple positions and that shows in advance what bone bridge between the medial tunnel and the lateral tunnel will be created, wherein a bone bridge indication displayed on the indicator is a function of a current position of the medial tunneler.

An example of a bone bridge planning indicator is, e.g., a set of markings (such as markings on a medial cannula) ranging from a minimal bone bridge up to a 3 cm bone bridge. Indication markers are on a medial cannula.

An example of using tool 1 is to perform bone-tunneling in shoulder surgery, comprising steps of constructing a medial tunnel using tool 1 that comprises guide body 11, medial tunneler 5 and top handle 2. Preferred examples of medial-tunnel-constructing are, e.g., a K wire process comprising forming the medial tunnel by use of a K wire, followed by sliding guide body 11 of surgical tool 1 over the K wire; a drill guide process comprising, with guide body 11 positioned as a drill guide, inserting an appropriately sized drill bit (not shown) through guide body 11 and drilling the medial tunnel; and a mallet action process comprising applying force to the top handle 2 of the surgical tool 1 to mallet the medial tunneler 5 in and thereby form the medial tunnel.

Tool 1 is useable to construct a tunnel through the to-be-repaired tendon, or, alternately, not through the to-be-repaired tendon.

Tool 1 is useable during repair of a rotator cuff in a vicinity of a humeral head. While tool 1 positions the rotator cuff, the rotator cuff is held by same tool 1 to the humeral head. For example, tool 1 is useable for piercing a to-be-repaired tendon with a pointed tip of an obturator, followed by pulling the pierced tendon to a desired location, followed by constructing a tunnel.

Advantageously tool 1 is useable for constructing a tunnel with a to-be-repaired tendon in place, and without usage of a soft tissue suture passer device.

Tool 1 is useable in a surgical tunneling method, comprising medial tunneling in a shoulder area of a patient, wherein a straight medial tunnel is formed by medial tunneler 5, followed by lateral tunneling in the shoulder area, wherein a lateral tunnel is formed by lateral tunneler 6. When tool 1 is used, advantageously a formed location of the medial tunnel after the tool 1 has been operated by the surgeon to form the medial tunnel matches a preplanned location that the surgeon intended the medial tunnel to define.

In a preferred example of operating tool 1, pin placement is used and medial tunneling by the medial tunneler 5 is at a place where a pin has been placed, before performing a medial tunneling step, wherein the pin represents where the surgeon operating tool 1 wants the medial tunnel to be formed.

In a preferred example of operating tool 1, a medial tunnel is formed by tool 1 in a place preselected by the surgeon before tunnel-formation, followed by the lateral tunnel being formed by tool 1 in a place preselected by the surgeon before tunnel-formation.

Another preferred example of using tool 1 is to form a medial tunnel by operation of the medial tunneler 5; and, after tool 1 has been rotated, form a lateral tunnel by operation of the lateral tunneler 6.

By adjusting depth of the medial tunnel and rotating tool 1, a location is selected of where the lateral tunnel will be formed.

Also tool 1 is useable to form a lateral tunnel and a medial tunnel during rotator cuff surgery, by forming the lateral tunnel via access that is a percutaneous needle stick and forming the medial tunnel via access that is a percutaneous needle stick.

Preferably tool 1 is used with an inventive suture passer kit comprising a suture retriever (such as, e.g. suture retriever 15 in FIGS. 20-20A) comprising a first member with a loop (such as, e.g., loop 15L in FIGS. 20-20A) at a distal end of the first member and a second member having assembled thereon a set of sutures, wherein the first member is sized to fit inside the lateral tunnel and the second member is sized to fit inside medial tunnel 5, and wherein the second member and/or the sutures thereon are pullable by the loop on the suture retriever.

Advantageously, use of suture anchors can be avoided. Regarding avoidance of suture anchors, see, e.g., S. Kuroda et al., “Advantages of Arthoscopic Transosseous Suture Repair of the Rotator Cuff without the Use of Anchors,” Clin Orthop Relat Res (2013) 471:3514-3522.

In another preferred use of tool 1, tunnel-forming is performed by tool 1, and also tool 1 is used to perform aspirating biologic material from where the tunnel-forming is performed, without needing to use a separate surgical tool for aspirating.

In a preferred example, tool 1 receives a withdrawal device (such as, e.g., a syringe) and holds the withdrawal device while biologic material is being received therein. This is useful, for example, for a syringe to aspirate bone marrow from a bone into which a tunnel is being formed by tool 1.

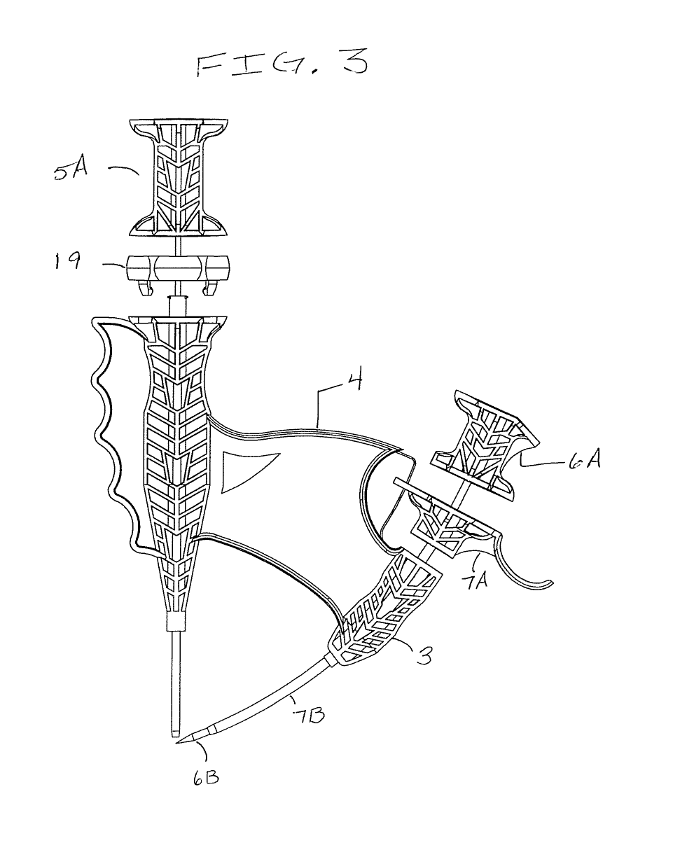

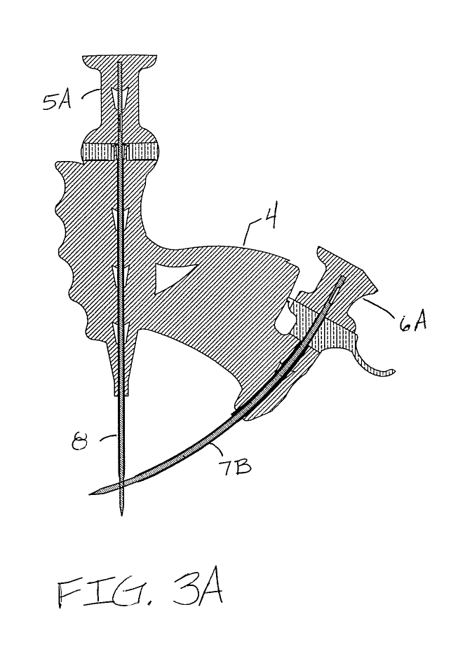

Another example of using invention tool 1 is to (1) position guide body 11 in a desired location and at a desired angle for a medial tunnel, (2) create the medial tunnel by drilling, by driving the guide pin, or by malleting in the medial obturator, (3) remove the medial drill, guide pin or obturator, (4) create a lateral tunnel by malleting in the lateral obturator, (5) remove the lateral obturator, (6) place a suture retriever in the lateral tunnel, (7) place a suture passer (such as, e.g., suture passer 16 (FIGS. 4-4B) in the medial tunnel, (8) remove the suture retriever (such as, e.g., suture retriever 15) to pull the suture through, (9) remove the tool 1, and (10) knot sutures.

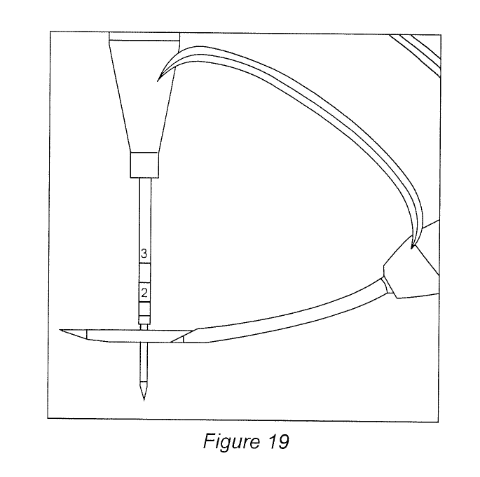

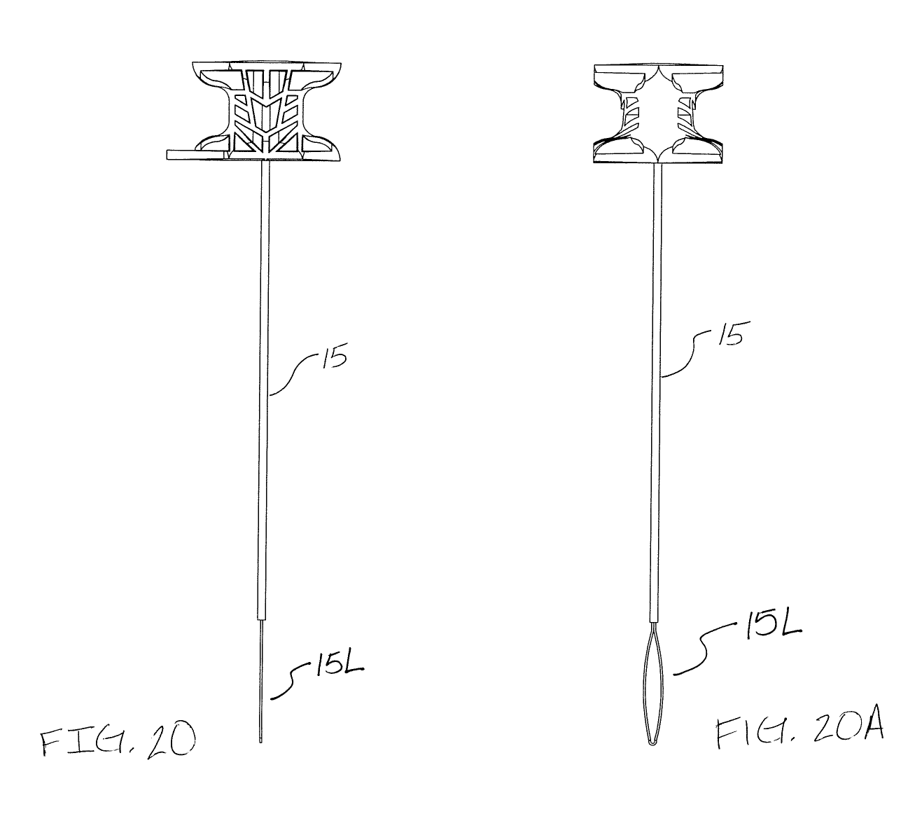

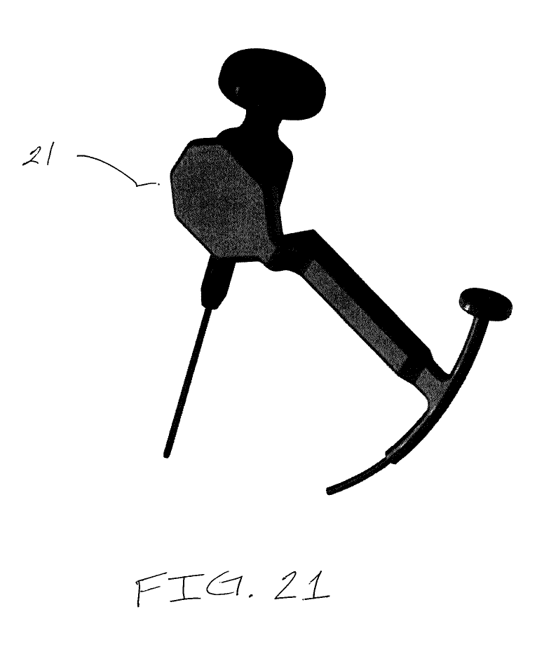

An inventive tool 1 is useable to create a medial tunnel first, before creating another tunnel. Tool 1 is useable for precise positioning and angling of a medial tunnel by a surgeon for aligning with a rotator cuff to be repaired. Depth of the medial tunnel can be adjusted by the user of tool 1 without removing tool 1, which correlates to the size of the bone bridge. Preferably tool 1 is provided with markings (such as the markings in FIG. 19) on the medial cannula wherein the markings on the medial cannula indicate approximate size of the bone bridge to aid the surgeon using tool 1 in positioning prior to creating the lateral tunnel.

When using inventive tool 1, the medial tunnel can be created by multiple approaches, such as, e.g., drilling, driving a guide pin, driving a K-wire, or with the medial obturator that is part of the instrument set.

Guide body 11/medial obturator of tool 1 allows positioning of the rotator cuff prior to creation of the medial tunnel. Using tool 1, the medial tunnel can be created through the rotator cuff so the surgeon doesn't have to relocate the tunnel after positioning the cuff. Tool 1 is useable to hold the rotator cuff in place until the suture has been passed through the cuff. Tool 1 is useable to pull the rotator cuff into position, eliminating the need to use a separate instrument to position and hold the rotator cuff. Tool 1 also eliminates the need for a soft tissue passer to get the sutures through the tendon.

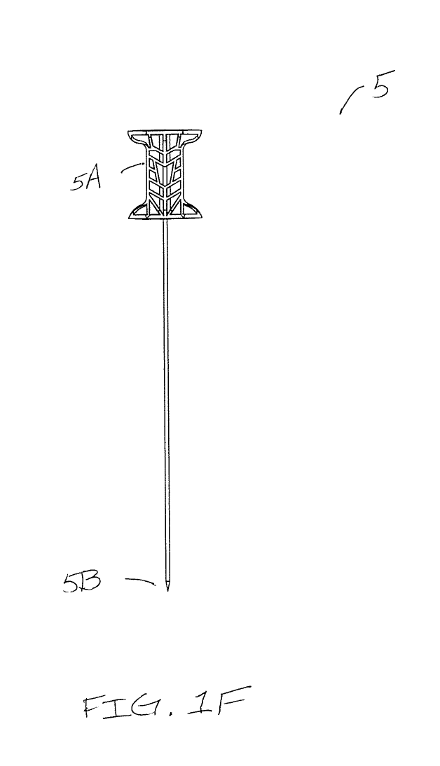

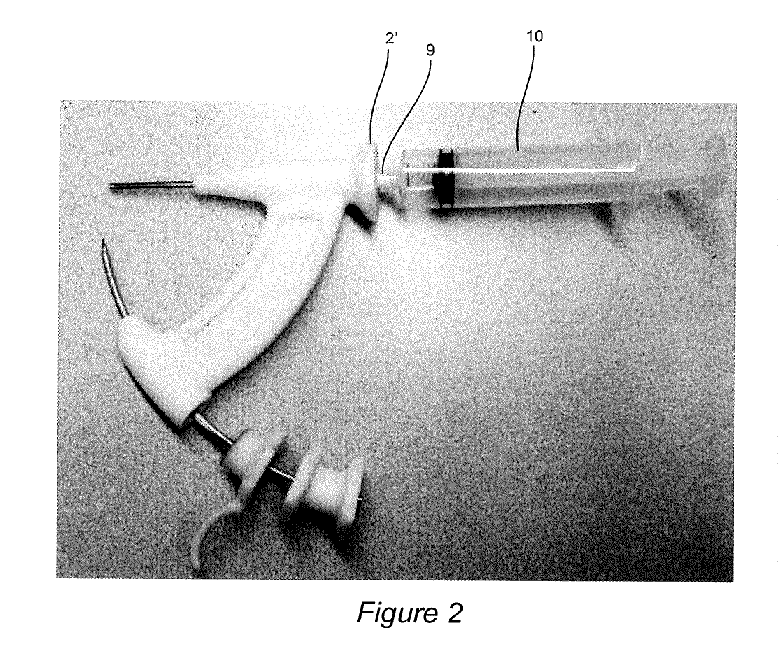

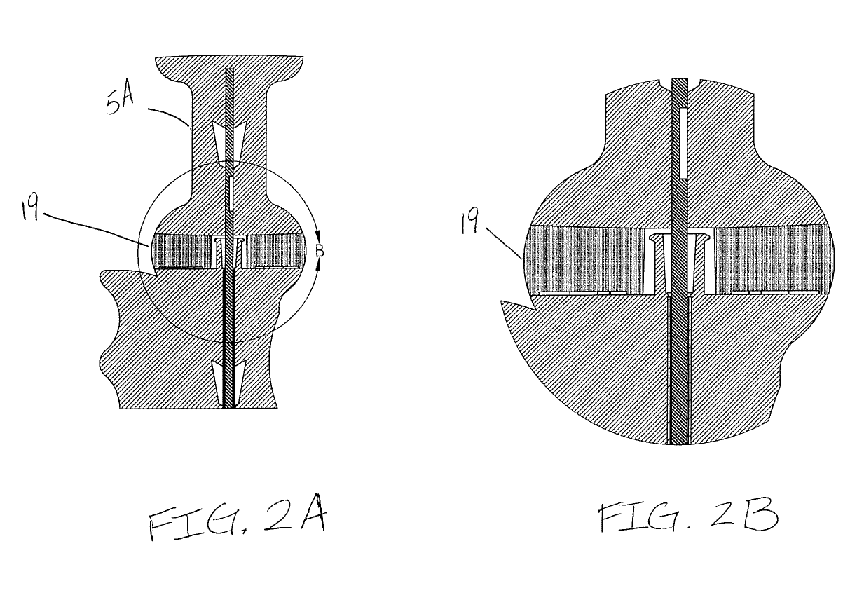

Preferably an inventive tool (such as tool 1) is provided with an integrated luer lock connection on the medial lumen for drawing and/or delivering biologics, thereby avoiding the need for separate instrumentation to aspirate or deliver the biologics to the surgical site. Luer lock collar 19 (FIGS. 1, 1D, 2A-2B) shields a luer lock (fluid) connection during malleting.

Guide body 11 of tool 1 acts as a drill guide for both medial and lateral tunnels. Through this feature, greater control of tunnel formation is provided, compared to commercially available surgical tools currently in use for rotator cuff surgery.

Tool 1 is characterized by a fixed guide body 11 design around a center of rotation of a lateral tunnel. In usage of tool 1, guide body 11 remains fixed while the surgeon creates the curved lateral tunnel.

In some preferred embodiments, a hinged lateral portion 21 (FIG. 21) is included. The hinged lateral portion 21 rotates, hinged around a pin (not shown in FIG. 21).

Guide body 11 of tool 1 allows the surgeon to locate the lateral tunnel at his/her discretion. Superior/inferior positioning is accomplished through varying depth of the medial cannula of tool 1. Anterior/posterior positioning is accomplished through rotating guide body 11 of tool 1 around the axis of the medial tunnel.

The lateral retractable cannula and lateral obturator of tool 1 are useable to create a lateral portal in the patient's shoulder. The lateral obturator of tool 1 pierces the skin while the lateral retractable cannula dilates the tissue. Such usage of tool 1 eliminates the need to create the portal with a scalpel, potentially reducing the size of the incision.

In rotator cuff surgery, alignment of the medial and lateral tunnels is wanted. Using tool 1, alignment of the medial and lateral tunnels is achievable as a function of tight fit between the lateral lumen and the lateral retractable cannula of tool 1, and the different bend radii of the lateral lumen and lateral retractable cannula. The structure of tool 1 preferably is a structure that creates single points of contact that maintain position of the cannula but reduces contact surface area and resulting friction to preserve smoother sliding action.

When constructing tool 1, preferably shaped tubing is used to provide tight anterior/posterior fit while allowing more float inferior/superior to reduce friction. Preferred examples of shapes for the shaped tubing are, e.g., oval and rectangular.

Preferably guide body 11 is constructed according to a geometry that facilitates alignment in the plane of the cannulas, to minimize anterior/posterior drift.

When constructing tool 1, preferably molded lips are included at either end of the lateral lumen, to create a tight fit at just those two locations.

Appropriately-sized tunnelers are used in tool 1. Preferably the lateral tunnel diameter is larger than the medial tunneler diameter, to allow for more tolerance in alignment while maintaining a small medial tunnel size. For suture passing, the medial tunnel should pass completely through the lateral tunnel. If the two tunnels are the same size, then their alignment must be perfect. As the lateral tunnel size increases, that difference in size is essentially acceptable tolerance on the alignment of the two tunnels.

Preferably a suture passer (such as, e.g., suture passer 16) is constructed to cooperate with tool 1. A suture passer is constructed so that sutures will be retained in the carrier, but be easily separable once the suture is passed through the bone tunnel. For example, preferably a plastic tube is used to construct a suture passer. In usage, the suture passer is placed in the medial tunnel. Examples of pulling out the suture passer are, e.g., a pulling-out method in which the suture retriever to capture the end of the suture passer and pull the entire suture passer through the bone tunnel and out the lateral side of guide body 11 of tool 1; a pulling-out method in which only a portion of the suture passer is pulled out through the bone tunnel along with the suture; a pulling-out method in which the suture is pulled through the tunnel, with the suture passer staying in the medial tunnel. An example of fixing a suture to the suture passer is a suture is fixed to the suture passer via a notch similar to side-load needles. In that example, a tight fit between the suture and the suture passer keeps the suture from slipping out prematurely; once passed, the user can easily remove the suture through the side-load.

Preferably the suture passer is loaded with sutures of multiple colors, to enhance suture management. When multiple sutures are used to repair a rotator cuff arthroscopically, if using the same color sutures, determining which ends of the suture belong to one another could be difficult. Preferably multiple (such as three) separate colors are combined in a suture passer kit, for quick and easy differentiation between individual sutures.

Another advantage of using tool 1 is that lateral tunnel size is provided that allows for desirable subcortical knot placement that avoids the problem observed when conventional surgical tools are used, that post-operative micro-motion of a suture can lead to wearing of bone. Tunnels can be created by use of tool 1 where the lateral tunnel created by the lateral retractable cannula is large enough to place the suture knots subcortically (i.e., below the surface of the bone). The tight fit of such knots resists the unwanted micro-motion and provides a larger contact area for the loads between the suture and bone, resulting in a lower probability of the suture wearing through the bone over time.

By “subcortical knot”, we mean a knot below the cortical layer of bone. Subcortical knots were invented by Brian Dross, US 20140107672 published Apr. 17, 2014. A subcortical knot is NOT an example of a suture anchor.

In the inventive methods, the medial tunneling and the lateral tunneling in some embodiments have locations, relative to the to-be-repaired tendon, of being through the to-be-repaired tendon. In other embodiments, the medial tunnel and the lateral tunneling have locations, relative to the to-be-repaired tendon, of being NOT through the to-be-repaired tendon.

In some embodiments, the inventive methods are performed relative to a damaged rotator cuff having a partial tear. In other embodiments, the inventive methods are performed relative to a damaged rotator cuff having a complete tear.

After tool 1 is used to create bone tunnel(s), preferably stitching according to the invention is performed. Examples of a stitch pattern formed when performing a stitching step of an inventive method are, e.g., a simple stitch pattern stitching a single tunnel; a box stitch pattern with inverted mattress stitch with knotless medial row stitching a set of two tunnels; a box stitch pattern with inverted mattress stitch with a locking medial row stitching a set of two tunnels; a standard X stitch pattern stitching a set of two tunnels; a boxed-X stitch pattern stitching a set of two tunnels; a stitch pattern stitching a set of three tunnels; etc.

When a stitching step according to the invention is performed, preferably no suture anchor is used. When a stitch pattern has been constructed according to the invention, preferably no suture anchor is present.

For construction of an arcuate tunnel according to the invention, an inventive tool or device preferably comprises an arcuate lumen. Preferably the arcuate lumen comprises a stylus (such as, e.g., a rigid stylus; a flexible stylus) protruding from the arcuate lumen. A rigid stylus protruding from the arcuate lumen is preferred for constructing an arcuate tunnel according to the invention.

Tool 1 is useable in curvilinear transosseous rotator cuff repair system surgical techniques, such as, e.g., performing full thickness repair with locking or knotless medial row fixation with option for single site biologic harvest and delivery; minimally invasive repair; suture management with stress reducing subcortical knots; use of preloaded multi-colored sutures and Transtendon Passing Loops.

The invention may be further appreciated with reference to the following non-limiting examples.

### EXAMPLE 1

A syringe 10 is attached to an inventive tool at handle 2′ (FIG. 2) at harvest port 9. Syringe 10 is attachable and detachable from handle 2′.

### EXAMPLE 2

In this inventive example, a guide kit comprises a guide body 11 and straight cannula; straight obturator; curved cannula; curved obturator; and suture retriever 15.

### EXAMPLE 3

In this inventive example, a Curvilinear Transosseous Rotator System simplifies and improves arthroscopic transosseous repair and saves time, provides rigid fixation, and provides multiple suturing options. Percutaneous streamlined tunneling is provided, which saves time and reduces incisions to small punctures. Transtendon UHMPE sutures are placed without needing secondary suturing devices. Bone bridges formed by the system of this example are robust. Extending past the Greater Tuberosity, tunnels are at maximum proportions to the patient. Suturing uses preloaded Transtendon Passing Loops for rapid Transtendon constructs. Inventive subcortical knots reduce stress concentration points and provide low profile fixation. Surgeons who wish to harvest humeral head bone marrow aspirate (BMA), can do so using the inventive system. BMA is capable of being drawn through a port during tunneling—avoiding need of a BMA Harvest Kit, associated surgical time and secondary operative sites.

### FULL THICKNESS ROTATOR TEARS

Full thickness tears vary in size and level of displacement/retraction. The surgeon first assesses if the cuff/tear mobility is sufficient for a repair. The surgeon also determines if the rotator cuff tear is suited for inventive “grasp-and-pierce”/“pierce-and-relocate” methods which combine tissue suturing and bone tunneling, or whether the tear is better addressed by passing sutures through the rotator cuff only after sutures are resting in bone tunnels.

In the inventive “grasp-and-pierce” and “pierce-and-relocate” methods, bone tunneling and tissue suturing are combined. Options of tissue suturing post tunneling are dependent on the type of secondary stitching device preferred by the surgeon. In this case, the tissue approximation steps are skipped the guide is positioned directly against bone, then stitches are passed after tunneling as would be the case with conventional suture anchors or other transosseous devices.

Use of the inventive system is preceded by the surgeon's preferred debridement protocol.

Tissue Approximation Step

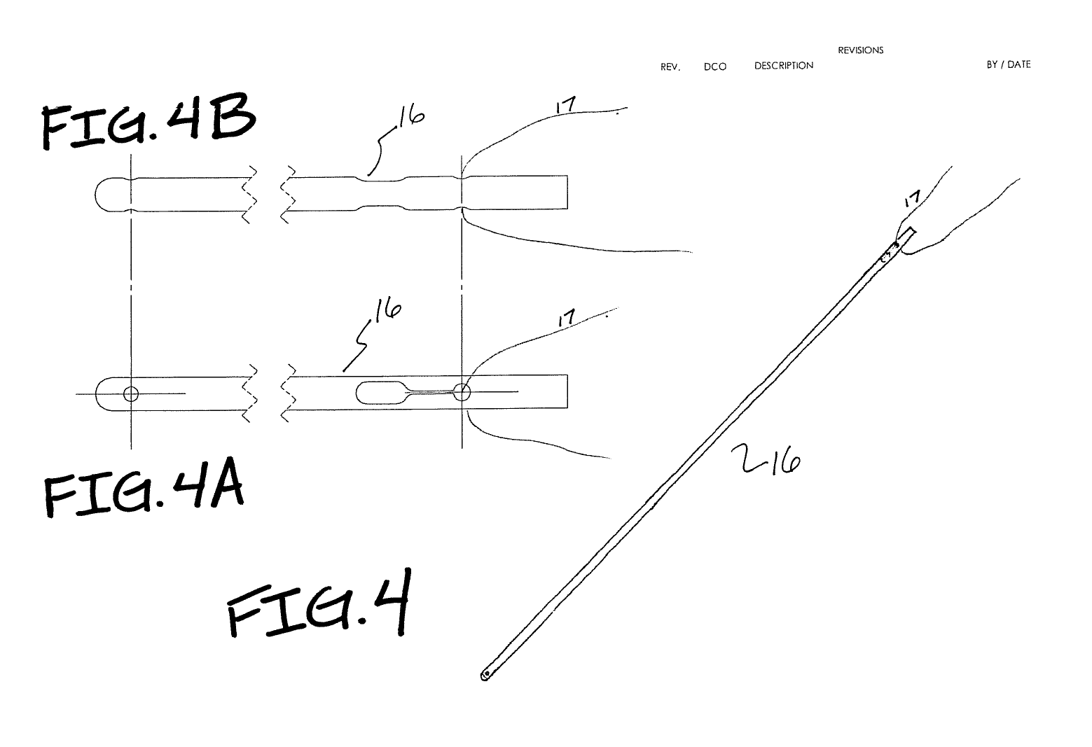

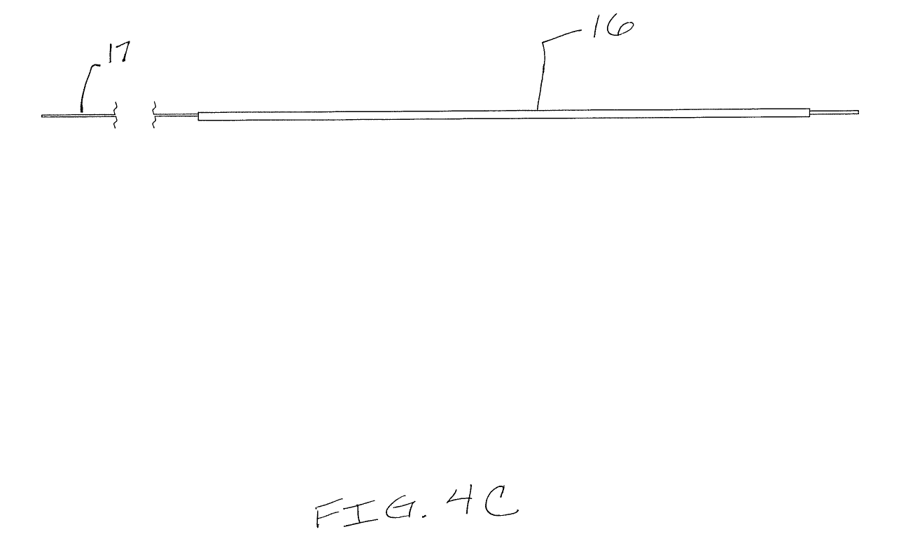

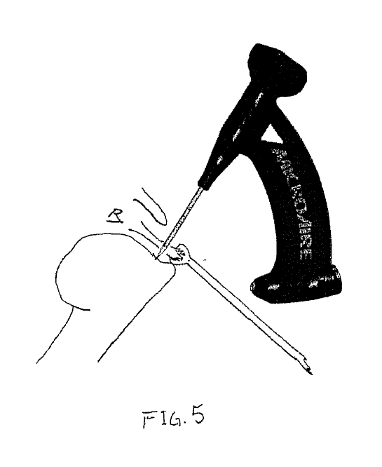

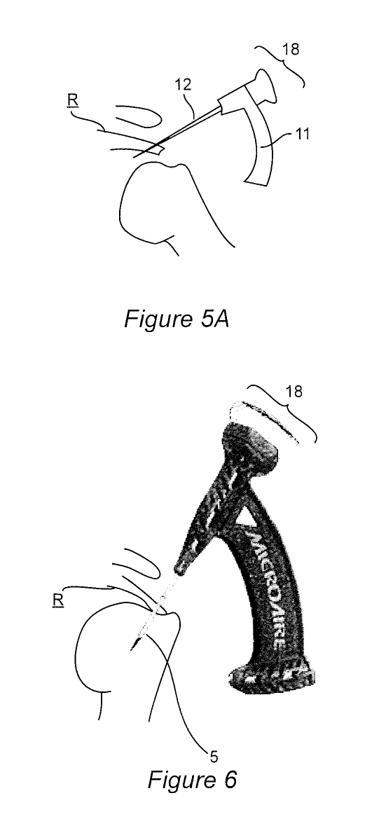

A “grasp-and-pierce” option (see FIG. 5) for performing the tissue approximation step is as follows: Through a standard lateral portal, rotator cuff R is mobilized then grasped. Then through the same portal or percutaneously, cuff R is pierced with the medial cannula to hold the cuff 4 in situ.

A “pierce-and-relocate” option (see FIG. 5A) for performing the tissue approximation step is as follows: Assemble the straight obturator 12 and the guide body 11 to construct assembly 18. Through a percutaneous puncture, cuff R is pierced with the assembled obturator. Then cuff R is levered into position.

Medial Tunnel Formation Step (see FIG. 6)

With the rotator cuff R correctly positioned, the assembly 18 is malleted to the desired calibration. (Note: the deeper the medial tunnel, the greater the bone bridge.)

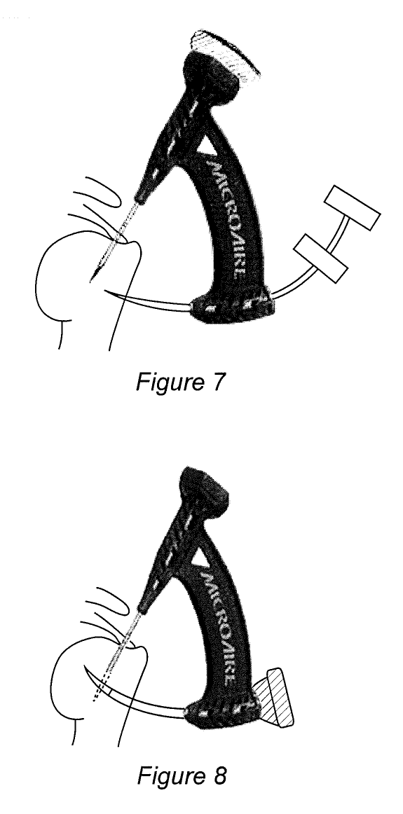

Lateral Tunnel Initiation Step (see FIG. 7)

Assembly: curved cannula 13 and curved obturator 14 are combined inserted into the guide body 11.

With the guide body 11 having the straight obturator 12 and cannula in the medial tunnel, the curved cannula 13 is pushed to bone, entering the lateral humeral cortex. Light malleting is performed if needed but without full insertion.

Disassembly: the medial obturator is removed. If the medial obturator is not removed, tunnels will not align.

In another embodiment, the suture retriever is placed, combined with the lateral obturator.

Lateral Tunnel Completion Step (see FIG. 8)

With the medial obturator removed, the curved cannula 13/obturator 14 is malleted until the caps contact the guide body 11.

Disassembly: ONLY the curved obturator 14 is removed, with no twisting, and with pulling following the curve. The cannula 13 is NOT removed.

In another embodiment, the suture retriever is placed with the lateral obturator, and only the lateral obturator is disassembled, leaving the suture retriever in place.

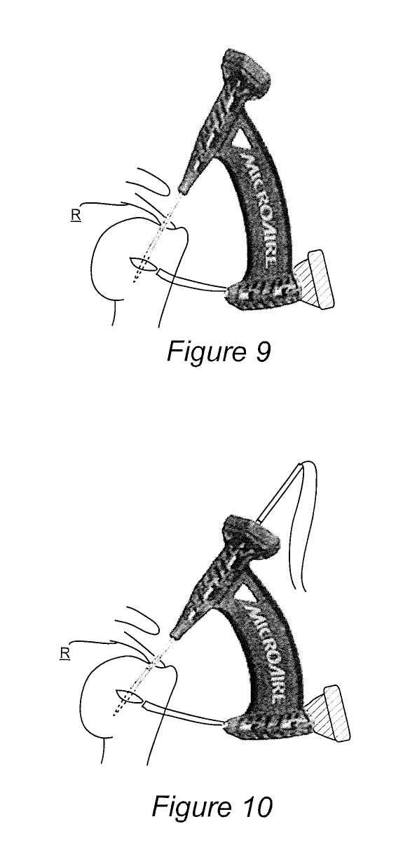

Suture Passing Preparation Step (see FIG. 9)

With the curved lateral obturator 14 removed and the curved cannula 13 in situ, the suture retriever is fully inserted into the lateral curved cannula 13.

Assembly: The suture retriever is inserted into the curved obturator 13/guide body 11.

Preferably an alignment feature is included in the hubs, to guide the wires to open.

Suture Loading/Passing Step (see FIG. 10)

Suture passer 16 that has been preloaded is selected, and then inserted to the calibration mark. Then suture retriever 15 is withdrawn and sutures are passed.

Assembly: preloaded suture passer 16 is inserted into the straight medial cannula/ guide body 11.

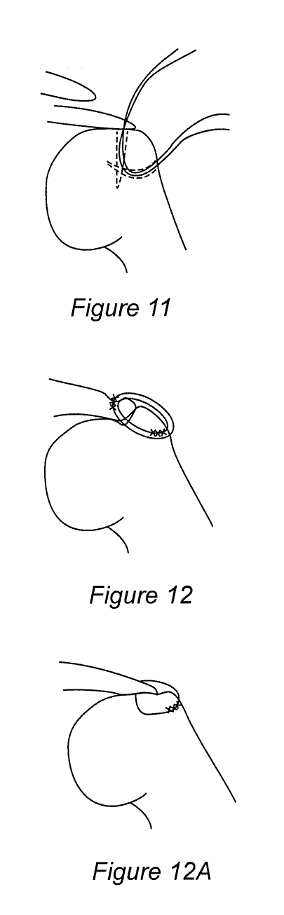

Component Removal Step (see FIG. 11)

Components are removed from the surgical site while ensuring that sutures are not pulled from tunnels:

A. Suture passer 16 is removed;

B. Lateral curved cannula 13 is removed, without twisting. The removal is by pulling, following curvature.

C. Guide body 11/medial cannula is removed with a twisting motion if needed.

### EXAMPLE 4

### Suture Management

Suture management is linked to the number of bone tunnels required, the stitch pattern desired and if the medial row is locked or knotless. To assist in a multitude of stitch patterns, preloaded suture combinations and colors are used, such as: two UHMPE sutures; three UHMPE sutures; one UHMPE suture and one Transtendon Passing Loop; two Transtendon Passing Loops; etc.

Stitch patterns to construct in this Example without the use of a secondary suture passer like an Arthrex® Viper® are:
1. Simple Stitch with Knotless Medial Row
2. Simple Stitch with Locking Medial Row
3. Box/Inverted Mattress Stitch with Knotless Medial Row
4. Box/Inverted Mattress Stitch with Locking Medial Row
5. Standard “X” Stitch with Knotless Medial Row
6. Standard “X” Stitch with Locking Medial Row
7. Boxed-X Stitch with Knotless Medial Row
8. Boxed-X Stitch with Locking Medial Row

A surgeon selects between stitch patterns for medical considerations, such as, for example, to select a stitch pattern that will provide more or less compressive force during healing.

### EXAMPLE 4A

### Suture Management/Knotless; Locking Stitches and Subcortical Knots

The stitch patterns in FIGS. 12-12A are for either locking or knotless medial rows.

FIG. 12 shows a locking medial row having a knot above the rotator cuff.

FIG. 12A shows a knotless medial row.

FIGS. 12-12A are examples of use of an inventive subcortical knot in the lateral cortex.

### EXAMPLE 4B

### Suture Management/Single Tunnel—Simple Stitch

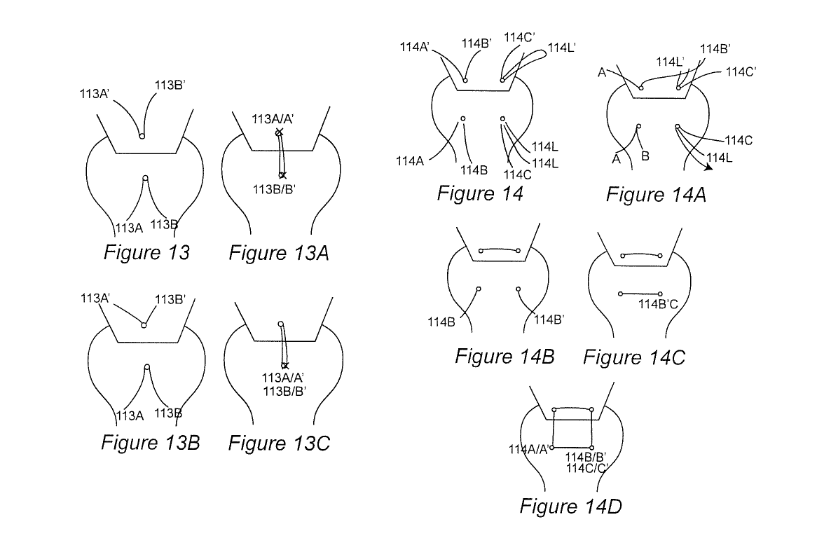

Within a single bone tunnel, any of the preloaded passers can be contained. However, if a patient's pathology requires only one bone tunnel, simple stitches are the easiest option by using a kit having two UHMPE of different colors. Sutures tied as 113A/113A′ and 113B/113B′ (FIGS. 13-13C) are either stacked within the lateral bone tunnel as subcortical knots, or one suture is over the medial tunnel and one is tied as a subcortical knot to create a locking medial row.

### EXAMPLE 4C

### Suture Management, Two Tunnels—Box/Inverted Mattress with a Knotless Medial Row

With two transosseous tunnels, a suture passer having two UHMPE sutures of different colors rests in one tunnel. And a kit having a suture of a third color and a Transtendon Passing Loop rests in the adjacent tunnel. Referring to FIGS. 14-14D, loop 114L is used to transfer 114B′ in a Transtendon approach. Suture ends are all tied beneath the cortex where one tunnel contains two stacked subcortical knots (114B/114B′ and 114C/114C′). No sutures are knotted over the rotator cuff tendon.

### EXAMPLE 4D

### Suture Management, Two Tunnels—Box/Inverted Mattress with a Locking Medial Row

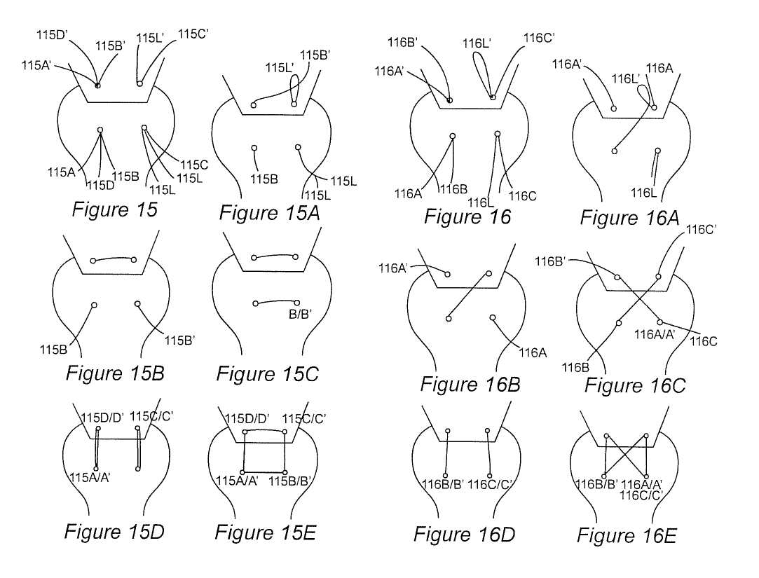

With two transosseous tunnels, a suture passer having three UHMPE sutures of different colors rests in one tunnel. And a suture passer having a suture and a Transtendon Passing Loop rests in the adjacent tunnel. Referring to FIGS. 15-15E, Loop 115L is used to transfer 115B′ in a Transtendon approach. Suture ends are all tied where knots rest both on the superior surface of the rotator cuff and within the lateral bone tunnels where 115B/B′ form a “box” shape.

### EXAMPLE 4E

### Suture Management, Two Tunnels—with Standard “X” Pattern

With two transosseous tunnels, a suture passer having two UHMPE sutures of different colors rests in one tunnel. And a suture passer having a suture of a third color and a Transtendon Passing Loop rests in the adjacent tunnel. Refering to FIGS. 16-16E, loop 116L is used to transfer 116A diagonally where 116A and 116A′ are tied in a subcortical “X” approach. Other suture ends are all tied beneath the cortex where one tunnel contains two stacked subcortical knots 116A/116A′, 116C/116C′. No sutures are knotted over the rotator cuff tendon when using the suture passing having two UHMPE sutures of different colors. If a locking medial row is needed, a suture passer having three UHMPE sutures is substituted for the suture passer having two UHMPE sutures of different colors.

### EXAMPLE 4F

### Suture Management, Two Tunnels—with Boxed-X Stitch Pattern

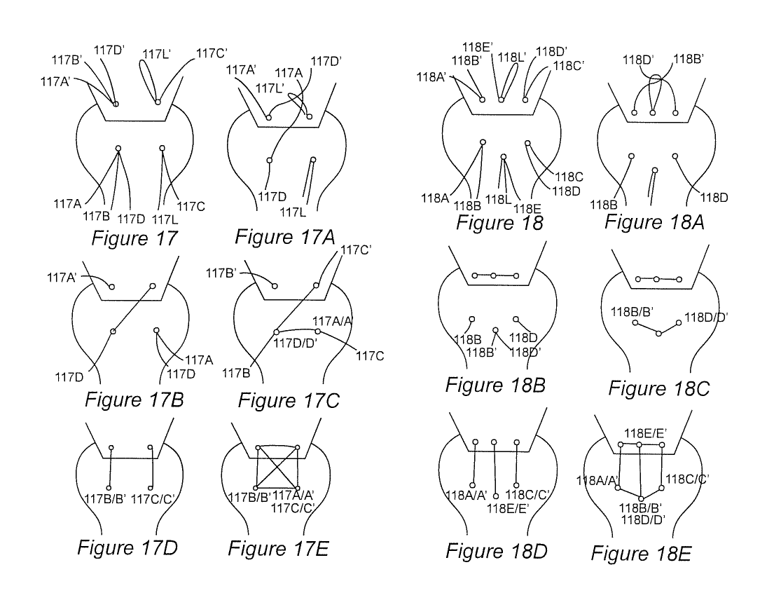

With two transosseous tunnels, a suture passer having three UHMPE sutures of different colors rests in one tunnel. A suture passer having a suture and a Transtendon Passing Loop rests in the adjacent tunnel. Referring to FIGS. 17-17E, loop 117L is used to transfer 117A and 117D′. 117A is transferred diagonally. 117D′ is transferred in a Transtendon approach. Suture ends are all tied and stacked as subcortical knots below. For a locking medial row, knots are not stacked and two are placed over the medial tunnels.

### EXAMPLE 4G

### Suture Management, Three Tunnels

With three or more transosseous tunnels, a combination of suture management options from Examples 4-4F are used. FIGS. 18-18E is a non-limiting example of a combination where a suture passer containing a suture and Transtendon Passing Loop is used in the center of three transosseous tunnels. A suture passer containing two Transtendon Passing Loops is also useable in some situations.

### EXAMPLE 5

### Tubular Suture Passer

In this example, the suture passer is a flexible plastic tube that has geometry at one end for the insertion and capturing of suture. To use, the suture passer is inserted into the medial cannula (also known as the straight cannula) of the guide body 11 after creation of the transosseous bone tunnel.

Alternate methods of pulling out the suture passer are as follows:

(1) A suture retriever (such as a suture retriever comprising a wire loop) captures the end of the suture passer and pulls the suture passer out of the lateral (curved) cannula. The suture is then separated from the suture passer, resulting in suture that is passed through the humeral head, which can then be used to repair the rotator cuff.

(2) Only a portion of the suture passer is pulled out, along with the suture.

(3) Only the suture is pulled through the tunnel, and the suture passer remains in the medial tunnel.

### EXAMPLE 5A

### Suture Passers

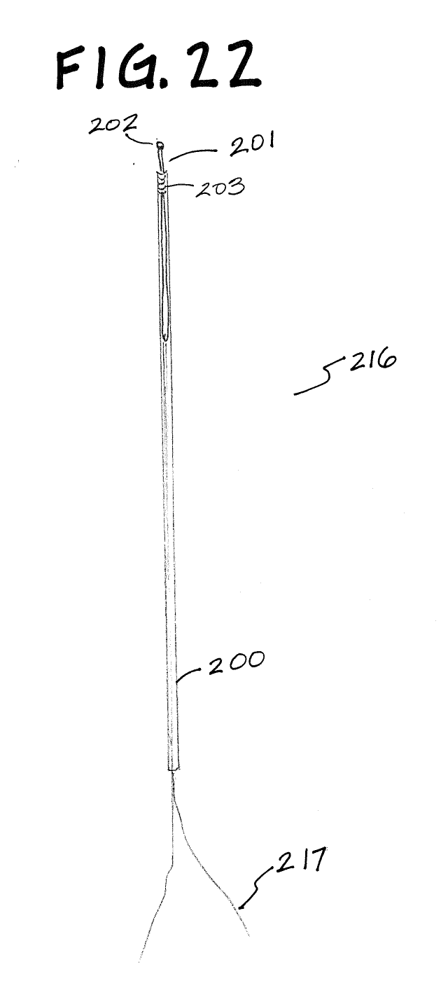

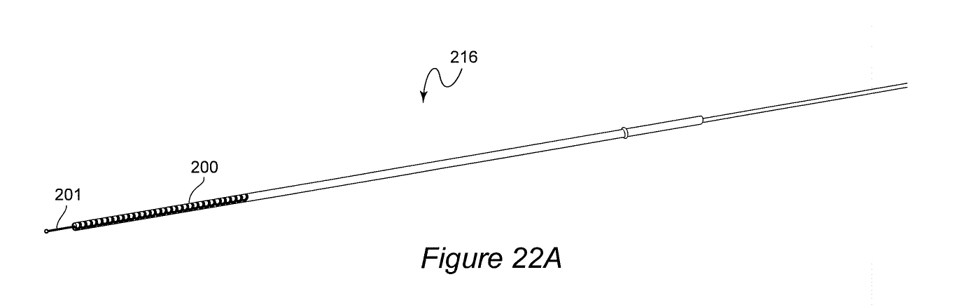

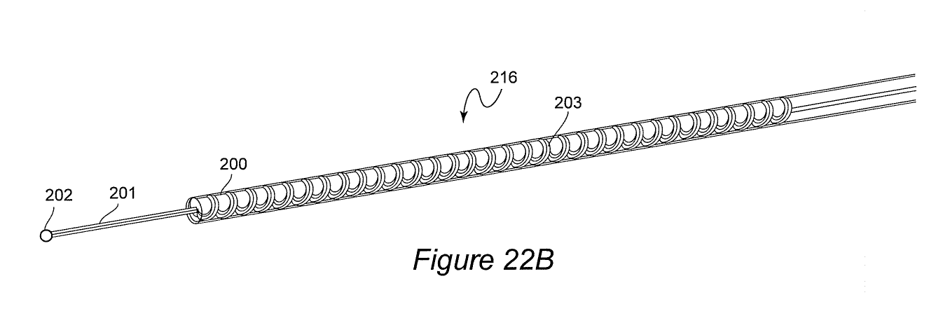

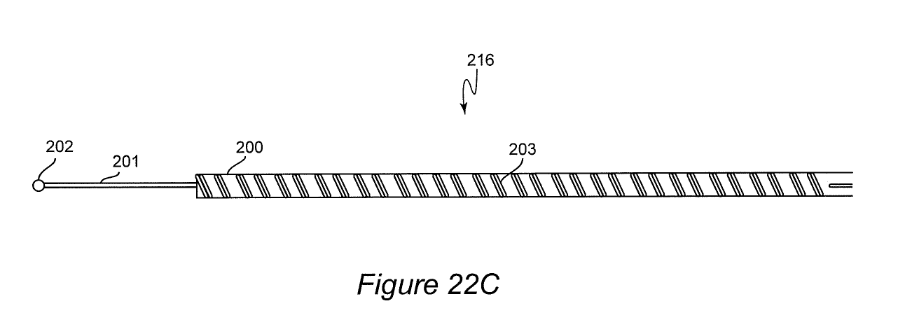

Inventive suture passer 216 (FIGS. 22-22C), threaded with suture 217, comprises an outer tube 200. Suture passer 216 comprises an inner wire loop 201.

Inner wire loop 201 comprises a knotted protruding tip 202 where inner wire loop 201 protrudes from outer tube 200. A capture mechanism is thereby provided. Round edges of knotted protruding tip 202 of suture passer 216 are conducive to connecting up with a loop of a suture retriever (such as, e.g., suture retriever 15). Suture passer 216 is easily removable in the event of a non-pass. Stiffness of inner wire loop 201 pushes through blood and debris to reduce failed passing.

Inner wire loop 201 comprises coiled wire 203. Preferably coiled wire 203 is disposed inside outer tube 200. A coil size of coiled wire 203 adds controlled friction, which reduces a chance of push-back and failed passing. The illustrated number of coils in coiled wire 203 in FIGS. 22 and 22A-22C respectively is illustrative and the number of coils is not particularly limited.

### EXAMPLE 6

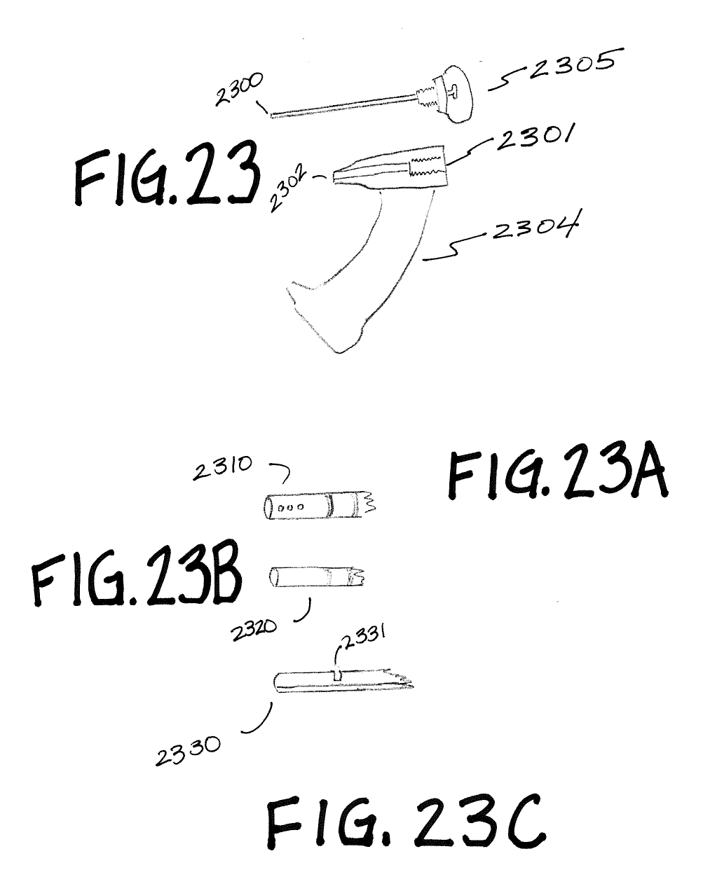

Medial cannula 2305 (FIG. 23) is removable from support member 2304. Tip 2300 of medial cannula 2305 enters support member 2304 at opening 2301 and is pushed inwards until tip 2300 exits opening 2302. Medial cannula 2305 comprises a distal luer connection.

Examples of configurations for tip 2300 are, e.g., tip 2310 (FIG. 23A); tip 2320 (FIG. 23B) and tip 2330 (FIG. 23C).

Tip 2310 is fenestrated, for increased BMA harvest efficiency.

Tip 2330 is occluded, for growth factor (e.g., BMAC, BMA, etc.) delivery. An open cut-away horizontal/bias slot at a specified depth is provided, to deliver growth factor between cuff and tendon. A recessed longitudinal slot 2331 acts somewhat like cannulation of orthopaedic screws. When using tip 2330, suture in situ acts as a guide wire; recessed slot 2331 acts as cannulation; and, user of tip 2330 finds the original medial bone tunnel after placing suture(s)/loop(s) to deliver the growth factors where needed most, namely, the bone-tendon interface.

### EXAMPLE 7

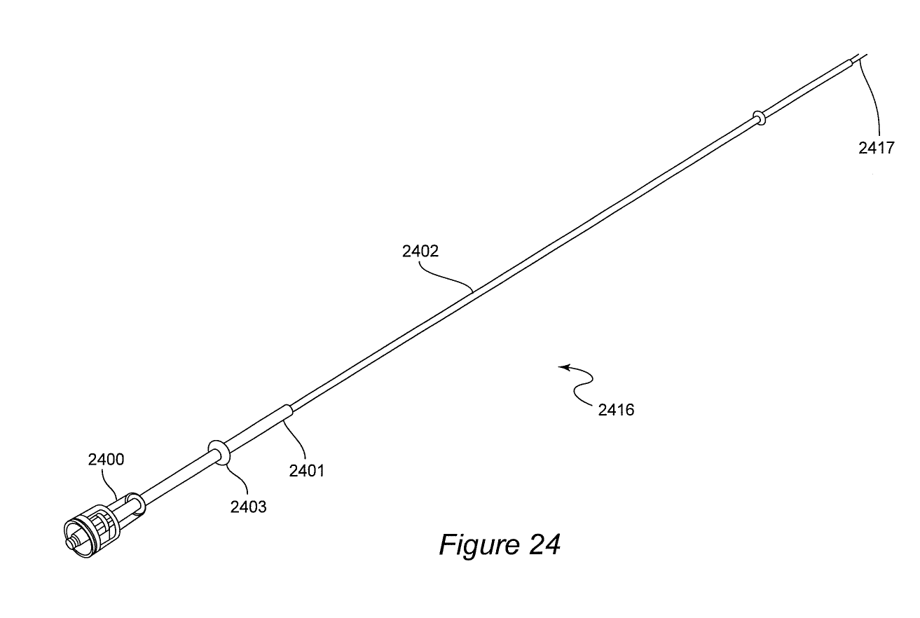

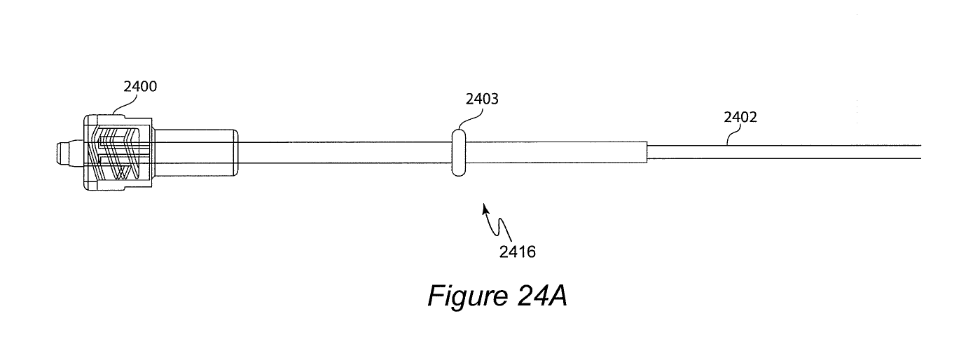

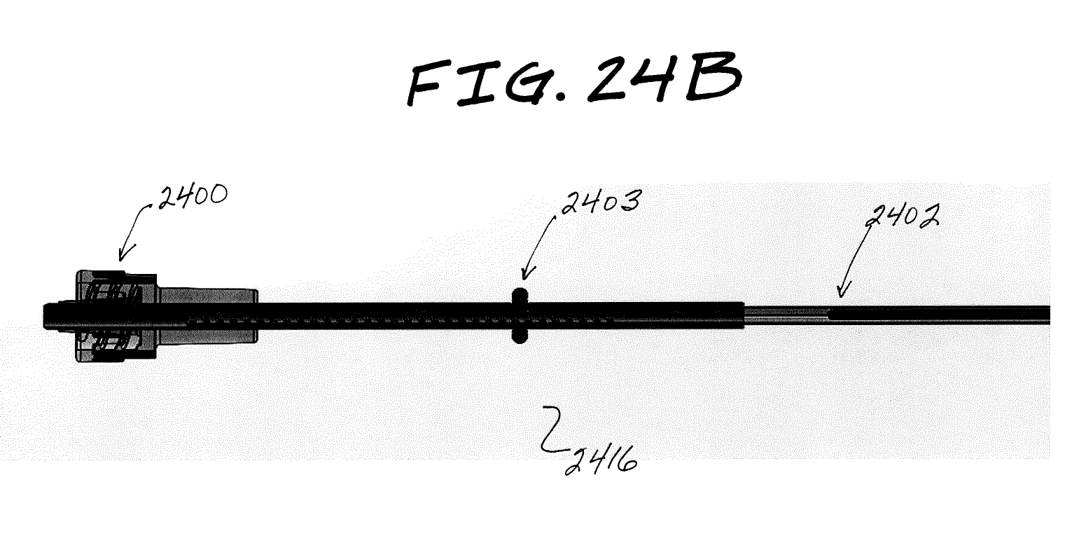

The inventive suture passer 2416 (FIG. 24), threaded with suture 2417, comprises luer tip 2400, cannula 2401, suture carrier tube 2402 and retaining ring 2403. Luer tip 2400/cannula 2401 at an end of suture carrier tube 2402 provide a means for attachment, provide strength, protect the tip of the suture carrier tube 2402, and align the suture 2417 with the medial cannula within the device body for a smoother interface.

Luer tip 2400 preferably is provided with internal threads by which suture passer 2416 is attachable to the device guide body.

Luer tip 2400 protects the wire tip of the suture/wire 2417, which is thin and can be easily bent/damaged from typical use if not protected by luer tip 2400. As shown in FIG. 24, advantageously that wire tip can remain within the metal cannula during all handling, eliminating risk of damage.

The metal cannula provides stiffness to the overall suture passer assembly, plus acts as a place to grip the suture passer for handling.

The luer tip 2400, by threading onto the device Guide Body, aligns the metal cannula with the medial cannula in the Guide Body, creating a smooth transition from suture passer to Guide Body when advancing the suture carrier tube 2402.

The suture carrier tube 2402 preferably comprises a flange that, at the proper depth, stops the tube's progression into the device.

### EXAMPLE 7A

When suture carrier tube 2402 (FIG. 24) is used as suture passer 216 (FIG. 22; Example 5A), luer tip 2400/cannula 2401 provides protection for the suture passer 216, and is a means of handling.

While the invention has been described in terms of its preferred embodiments, those skilled in the art will recognize that the invention can be practiced with modification within the spirit and scope of the appended claims.

1. A suture passer kit, comprising:
a suture retriever comprising a first member with a loop at a distal end of the first member;
a second member having assembled thereon a set of sutures, wherein the second member is sized to fit inside a medial tunnel, and wherein the second member and/or the sutures thereon are pullable by the loop on the suture retriever.

2. The suture passer kit of claim 1, wherein the second member is a suture passer that comprises an outer tube and an inner wire loop.

3. The suture passer kit of claim 2, wherein the inner wire loop comprises a knotted protruding tip where the inner wire loop protrudes from the outer tube.

4. The suture passer kit of claim 2, wherein the inner wire loop comprises coiled wire.

5. The suture passer kit of claim 1, wherein the second member is a suture passer that comprises a luer tip.

6. The suture passer kit of claim 5, wherein the suture passer further comprises a cannula.

7. The suture passer kit of claim 5, comprising a luer tip and a cannula at an end of a suture carrier tube.

8. The suture passer kit of claim 5, wherein inside the luer tip are internal threads.

9. The suture passer kit of claim 1, wherein the second member comprises a suture carrier tube inside which is disposed a flange.

10. The suture passer kit of claim 1, wherein the first member comprises an elongated member and the second member comprises an elongated member.

11. The kit of claim 1, wherein the first member comprises a tube and the second member comprises a tube.

12. The kit of claim 1, wherein the loop comprises a wire loop.

13. The suture passer kit of claim 1, wherein the second member is pullable by the loop on the suture retriever when the loop captures the suture passer at an intersection where the medial tunnel intersects a lateral tunnel.

14. The kit of claim 1, wherein the first member is a plastic tube and the second member is a plastic tube.

15. The suture passer kit of claim 1, wherein the set of sutures includes at least two different-colored sutures.

16. The suture passer kit of claim 1, wherein the set of sutures includes at least three different-colored sutures.

17. The kit of claim 1, in which suture anchors are not contained nor used with the kit.

18. A surgical stitching method during rotator cuff repair, comprising:
surgically constructing a set of tunnels, followed by
stitching without use of suture anchors and comprising use of a preloaded suture passer kit.

19. The surgical method of claim 18, wherein the set of tunnels is selected from the group consisting of: one tunnel; two tunnels; three tunnels.

20-22. (canceled)

23. A surgical stitch pattern, comprising:
a set of sutures defining a boxed-X shape, wherein the sutures stitch a surgical site on a patient.

24-57. (canceled)
